(19) United States
(12) Patent Application Publication
(10) Pub. No.: US 2024/0035650 A1
(43) Pub. Date: Feb. 1, 2024

(54) MULTIDIRECTIONAL ADJUSTABLE SUPPORT STRUCTURE

(71) Applicant: OXTI Pte Ltd, Singapore (SG)

(72) Inventor: Chih-Feng Ho, Singapore (SG)

(21) Appl. No.: 17/876,550

(22) Filed: Jul. 29, 2022

(51) Int. Cl. F21V 21/32; F16M 11/40; F16M 13/02; F21V 21/088

(52) U.S. Cl. CPC F21V 21/32; F16M 11/40; F16M 13/022; F21V 21/088

(57) ABSTRACT

A multidirectional adjustable support structure includes: a lamp; a tubular body, which is connected to the lamp and is provided with a spherical body; and a base, which includes an extension plate. The extension plate is formed with a cavity. The spherical body is rotatably received in the cavity. The tubular body includes a flexible tube. The base is provided with a clamping member. The spherical body being rotatably received in the cavity makes the cavity a rotation support point for the spherical body takes so as to allow the tubular body to rotate relative to the base, thereby allowing the lamp to randomly rotate relative to the base. The tubular body being a flexible tube makes it possible to bend and stretch in an arbitrary angle when in use and to contract and reduce when not in use, in order to reduce the space occupied thereby.

### TECHNICAL FIELD OF THE INVENTION

The present invention relates generally to an adjustable support, and more particularly to a multidirectional adjustable support structure of a lamp.

### DESCRIPTION OF THE PRIOR ART

Reference is made to Chinese Patent No. CN206880873U, which discloses a height-adjustable ultraviolent sterilization lamp, which comprises a lamp housing, a controller, an extendible rod, a weighted base, a reflective hood, and a fastening knob and is characterized in that the lamp housing is fixed on a curved tube; the curved tube is mounted, by means of the fastening knob, on the extendible rod; the controller is arranged on a middle of the extendible rod; the extendible rod is arranged on a sleeve; the sleeve is fixed on the weighted base; the weighted base is provided, on four corners of an underside, with universal rollers; the reflective hood is mounted on the lamp housing; leftward and rightward ends of the reflective hood are both provided with an ultraviolet light tube retaining seat; and an ultraviolet light tube is mounted in the ultraviolet light tube retaining seats.

However, operation of the known device adopts the free extendibility of the extendible rod and a height of the ultraviolet sterilization lamp is adjusted according to a practical need, and the fastening knob is arranged on the curved tube of the lamp housing and the extendible rod to adjust an illumination angle of the ultraviolet sterilization lamp. The known device allows only for height adjustment, and an inclination angle of the ultraviolet lamp is adjusted by means of the fastening knob in order to change the illumination angle, this making an illumination range very limited and thus hard to suit users' needs in different environments. Thus, there is still need for further improvement.

### SUMMARY OF THE INVENTION

In view of the above problems in association with the installation and mounting of the prior art device, the present invention aims to provide a multidirectional adjustable support structure.

To achieve the above objective, the present invention provides a multidirectional adjustable support structure, which comprises: a lamp; a tubular body, which is connected to the lamp and is provided with a spherical body; and a base, which is provided with an extension plate, the extension plate being formed with a cavity. The spherical body is rotatably received in the cavity.

In the present invention, the tubular body can be a flexible tube, and the spherical body is integrally formed with or is assembled on the tubular body.

In the present invention, the tubular body may comprise a connecting member, and the connecting member is connected to the lamp by means of a welding structure, a screwing structure, an adhesive bonding structure, or a magnetic attraction structure.

In the present invention, the cavity may comprise a spherical socket.

In the present invention, the base may comprise a clamping member, and the clamping member comprises a curved spring plate.

By having the spherical body rotatably received in the cavity, the spherical body may take the cavity as a rotation support point to allow the tubular body to rotate relative to the base to thereby allowing the lamp to rotate relative to the base. The tubular body being a flexible tube makes it to bend and stretch in an arbitrary angle when in use and to contract and reduce when not in use, in order to reduce the space occupied thereby.

### BRIEF DESCRIPTION OF THE DRAWINGS

FIG. 1 is a schematic view showing a multidirectional adjustable support structure according to the present invention.

FIG. 2 is a perspective view showing the multidirectional adjustable support structure according to the present invention.

FIG. 3 is a schematic view demonstrating, in the present invention, a spherical body is rotatable about a rotation support point defined by a socket relative to a base.

FIG. 4 is a schematic view demonstrating, in the present invention, a tubular body is bendable and stretchable in any arbitrary direction.

FIG. 5 is a schematic view showing the multidirectional adjustable support structure of the present invention applied to use with a height-adjustable chair.

FIG. 6 is a block diagram of a lamp in the present invention.

FIG. 7 is a schematic view demonstrating the lamp is controllable to emit light in a wireless manner.

FIG. 8 is a schematic view demonstrating the lamp is controllable to emit light in a wired manner.

### DETAILED DESCRIPTION OF THE PREFERRED EMBODIMENT

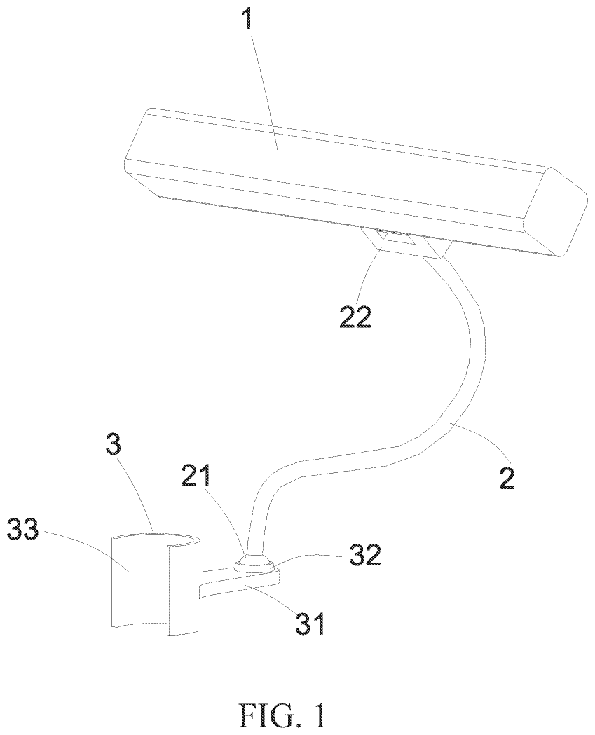

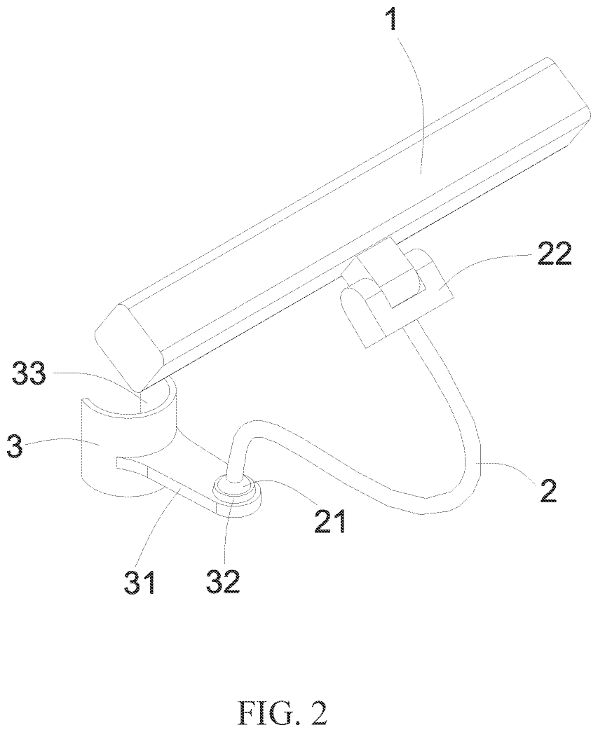

Referring to FIGS. 1 and 2, the present invention provides a multidirectional adjustable support structure, which comprises: a lamp 1, a tubular body 2, and a base 3.

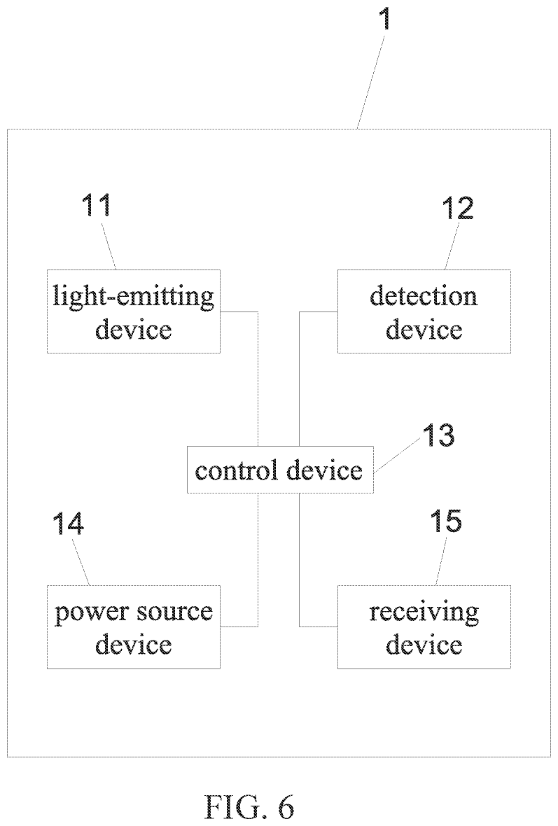

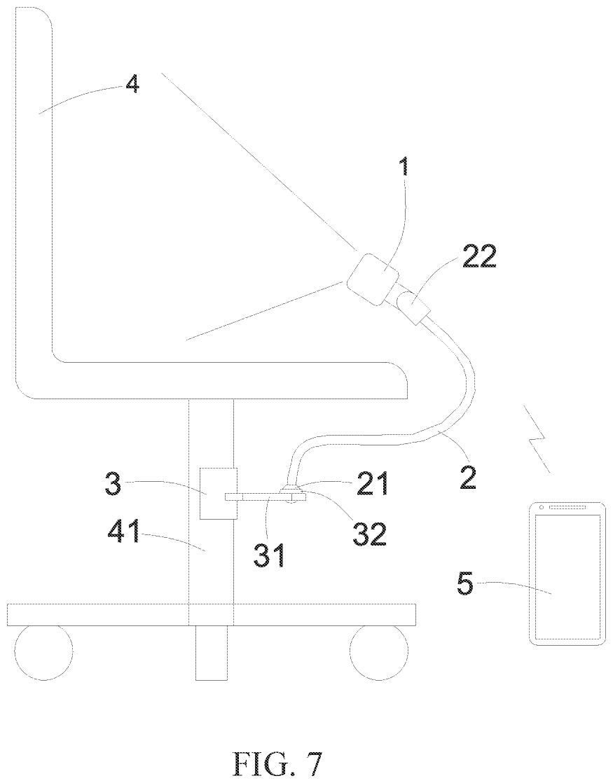

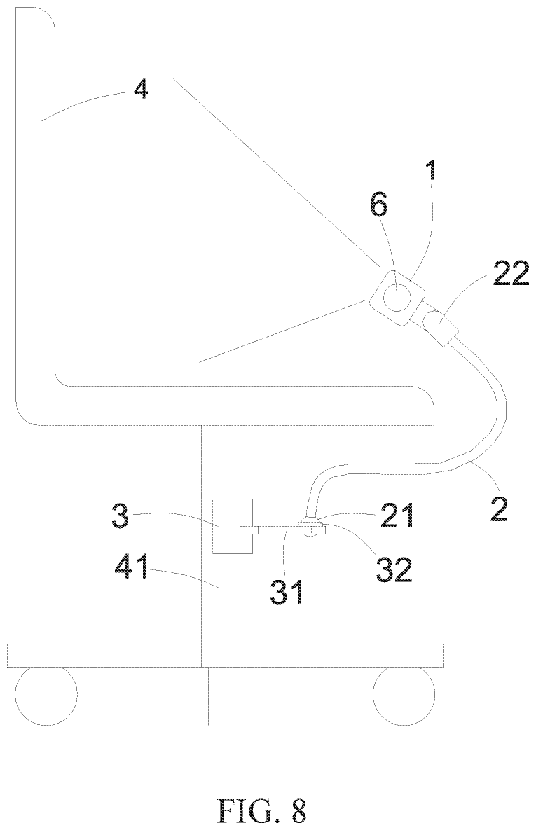

The lamp 1 can be for example a UVB (Ultraviolet Radiation B) lamp, a UVC (Ultraviolet Radiation C) lamp, a mercury lamp, a metal halide lamp, a xenon lamp, or other electronic lighting device that fulfills functionality of sterilization and disinfection. Referring to FIG. 6, specifically, the lamp 1 may comprise a light-emitting device 11, a detection device 12, a control device 13, a power source device 14, and a receiving device 15. The light-emitting device 11 can be a light bulb, a light string, or a light-emitting diode (LED) light, which serves as a light source of the lamp 1. The detection device 12 can be optical sensor device or an infrared sensor device, which is operable to sense or detect a human body. The control device 13 can be a central processing unit or a microcontroller, which is operable to control the light-emitting device 11. The power source device 14 can be a battery or other power supply arrangements. The receiving device 15 can be a wireless signal receiver or a wired signal receiver, and the receiving device 15 is operable to receive a wireless signal or a wired signal, so that the control device 13 may operate according to or in response to the wireless signal or the wired signal to control the light-emitting device 11. The wireless signal can be supplied from a smart phone 5 (shown in FIG. 7) or a remote controller, in order to control the lamp 1 to emit light in a wireless manner; the wired signal can be supplied from a wired switching device 6 (shown in FIG. 8), in order to control the lamp 1 to emit light in a wired manner.

The tubular body 2 is connected to the lamp 1 and is provided with a spherical body 21. Specifically, the tubular body 2 is a flexible tube, and the spherical body 21 is integrally formed with or is separately formed and assembled on the tubular body 2. The flexible tubular body can be formed of for example hydrated silica or silica gel, and has an elastic property, excellent deformability, and stable material characteristics and is bendable or flexible in an arbitrary angle and stretchable or contractable. The tubular body 2 is also provided with a connecting member 22, and the connecting member 22 is connectable to the lamp 1 by means of a magnetic attraction structure, a snap-fitting structure, a screwing structure, or an adhesive bonding structure. For example, the lamp 1 may be provided with a powerful magnet, and the connecting member 22 is provided with a structure that is magnetically attractable by the powerful magnet (for example, the connecting member 22 being itself made of a ferrous product). Or, in an alternative example, the lamp 1 is provided with a snap hook, and the connecting member 22 is formed with a snap opening. Or, in an alternative example, the lamp 1 and the connecting member 22 are each formed with a screw hole, and are fastened together by means of a bolt matching the screw holes.

The base 3 comprises an extension plate 31, and the extension plate 31 is formed with a cavity 32, and the spherical body 21 is rotatably received in the cavity 32. Specifically, the cavity 32 is a spherical socket, and sphere is the preferred shape, yet it is not limited to a spherical shape. The base 3 is also provided with a clamping member 33, and the clamping member 33 can be of a curved or arc spring plate, yet clips or clamps or other types are also usable. The spherical body 21 is fit into and embedded in the cavity 32, and the spherical body 21 is rotatable relative to the cavity 32. The spherical body 21 has an outside diameter that is slightly larger than an inside diameter of the cavity 32 in order to have the spherical body 21 securely retained in the cavity 32 without undesired separation therefrom.

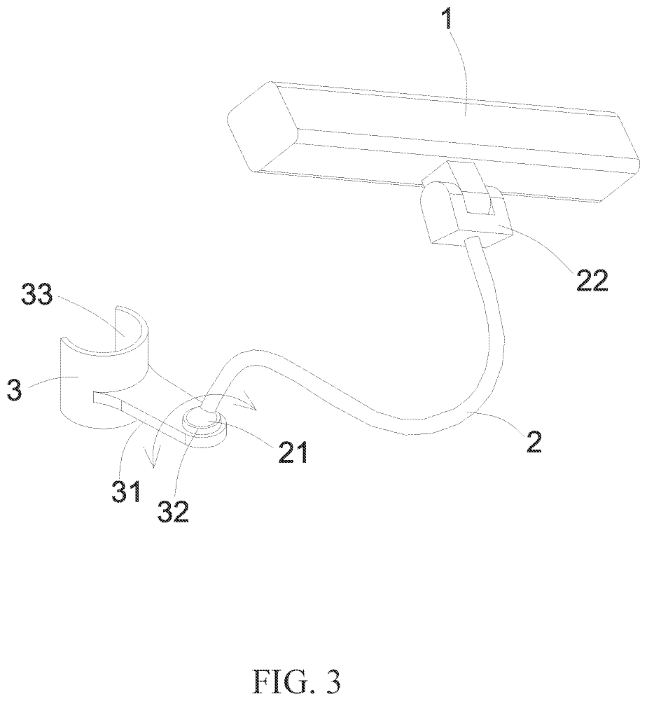

Referring to FIG. 3, the spherical body 21 is rotatably received in the cavity 32, so that the spherical body 21 may take the cavity 32 as a rotation support point to allow the tubular body 2 to rotate relative to the base 3 so as to realize relative rotation of the lamp 1 with respect to the base 3 either upwards, downwards, leftwards, and rightwards, for unlimited free rotation therebetween.

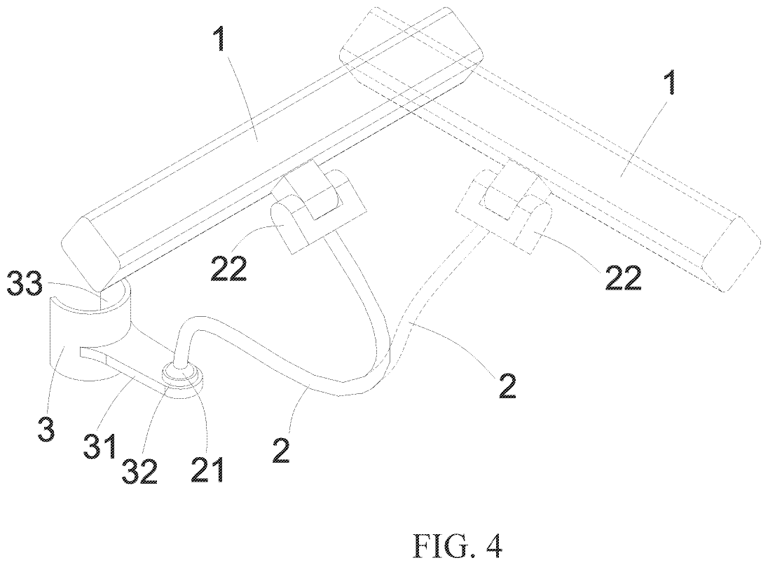

Referring to FIG. 4, the tubular body 2 is arranged between the lamp 1 and the base 3, and the tubular body 2 comprises a flexible tube, which, when used, can be bent in an arbitrary direction and can be stretched, and when not used, can be contracted to reduce the amount of space occupied thereby.

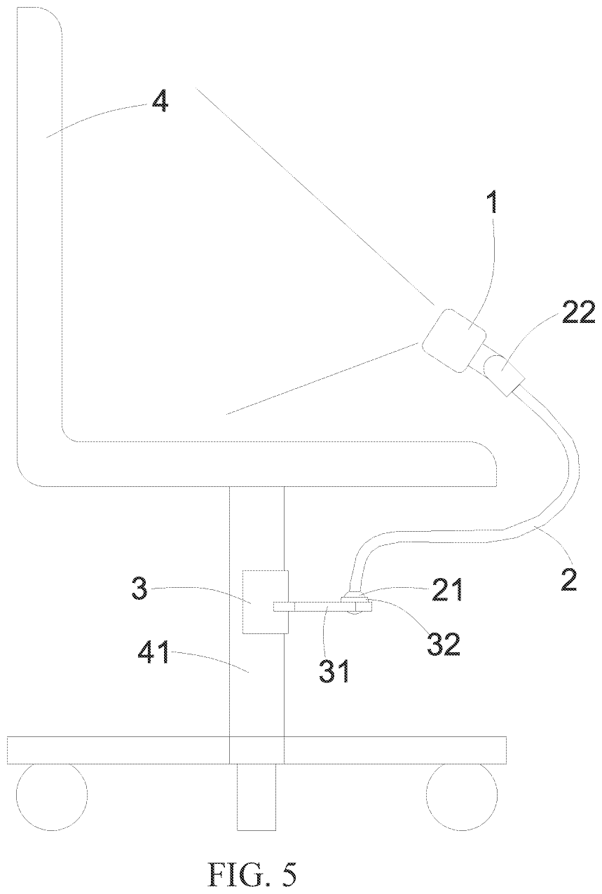

Referring to FIG. 5, the clamping member 33 of the base 3, if made in the form of a curved spring plate, can clamp on a cylindrical post or bar. For example, the multidirectional adjustable support structure according to the present invention is applicable to use with a height-adjustable chair 4, wherein the base 3 can be attached, through clamping with the clamping member 33, to a lift bar 41 of the height-adjustable chair 4, and except for the lamp 1 being rotatable relative to the base 3, the tubular body 2 is also manipulatable for bending and stretching in any arbitrary angle in order to fulfill adjustment in multiple directions for carrying out operations of sterilization and disinfection. For example, the lamp 1 may illuminate the height-adjustable chair 4 from an underside of the height-adjustable chair 4 toward a top side, or the lamp 1 may illuminate the height-adjustable chair 4 from one lateral side of the height-adjustable chair 4, and when not in use, the tubular body 2 can be reduced or contracted to allow the lamp 1 to be concealed under the height-adjustable chair 4 so as to reduce the amount of space occupied thereby.

Referring to FIGS. 6 and 7, after electrical power is supplied from the power source device 14 of the lamp 1, the detection device 12 continuously detect surroundings of the chair 4, so that when a person is detected approaching the chair 4, the detection device 12 signals the control device 13, and the control device 13 controls the light-emitting device 11 to stop emitting light in order to prevent hurting the person. When it is detected that no person is around the chair 4, the detection device 12 signals the control device 13, and the control device 13 controls the light-emitting device 11 to emit light so that the chair 4 can be sterilized by means of the light emitting from the lamp 1. Referring to FIGS. 6 and 7, the lamp 1 can be controlled in a wireless manner to emit the light, and in other words, the smart phone 5 or the remote controller is operated to supply a wireless signal to the receiving device 15 to allow the control device 13 to control, according to or in response to the wireless signal, the light-emitting device 11 to emit light. Referring to FIGS. 6 and 8, or alternatively, a wired manner may be adopted to control the lamp 1 to emit light, and in other words, the lamp 1 is provided with a wired switching device 6, and the wired switching device 6 is electrically connected to the receiving device 15, wherein the wired switching device 6 supplies a wired signal, and the receiving device 15 receives the wired signal to make the control device 13 to control, according to or in response to the wired signal, the light-emitting device 11 to emit light.

Based on the above structure, the present invention may achieve the following advantages:
(1) The present invention, when not in use, can be collapsed or reduced or contracted and concealed so as not to occupy much space and thereby leaving more space to be utilized by a user.
(2) The present invention is not subjected to constraints by the surrounding environment and only needs to attach to and fix to a support bar or pillar, and therefore is applicable to use in various environments.
(3) The present invention allows a user to do adjustment in multiple angles for achieving an optimum or most suitable angular position, and exhibiting extremely high flexibility, and enabling rotation in multiple angles to eliminate any dead zone in the illumination range.

I claim:

1. A multidirectional adjustable support structure, comprising:
a lamp;
a tubular body, which is connected to the lamp and is provided with a spherical body; and
a base, which comprises an extension plate, the extension plate being formed with a cavity, wherein the spherical body is rotatably received in the cavity.

2. The multidirectional adjustable support structure according to claim 1, wherein the tubular body comprises a flexible tube, and the spherical body is integrally formed with or is assembled on the tubular body.

3. The multidirectional adjustable support structure according to claim 1, wherein the tubular body comprises a connecting member, and the connecting member is connected to the lamp by means of a magnetic attraction structure, a snap-fitting structure, a screwing structure, or an adhesive bonding structure.

4. The multidirectional adjustable support structure according to claim 1, wherein the cavity comprises a spherical socket.

5. The multidirectional adjustable support structure according to claim 1, wherein the base comprises a clamping member, and the clamping member comprises a curved spring plate.
